(19) United States
(12) Patent Application Publication
(10) Pub. No.: US 2004/0186522 A1
(43) Pub. Date: Sep. 23, 2004

(54) Apparatus for pericardial augmentation

(76) Inventor: Leslie A. Beben, Sumpter, SC (US)

Correspondence Address: Richard C. Emery, 4189 Lakewood Avenue, White Bear Lake, MN, 55110-3925, US

(21) Appl. No.: 10/765,639

(22) Filed: Jan. 27, 2004

Related U.S. Application Data
(60) Provisional application No. 60/442,480, filed on Jan. 27, 2003.

(51) Int. Cl. A61N 1/36

(52) U.S. Cl. 607/9; 607/129

(57) ABSTRACT

A pericardial augmentation apparatus and method for electrically assisting the normal contraction of the heart. The invention comprises placing a mesh material apparatus over the heart within the pericardial space. Embedded in the apparatus is a plurality of electrode nodules that are synchronized to stimulate and assist in the normal contraction of the heart. The mesh of the device is adaptable to fit any size or shape heart and can be self-secured or sutured to the pericardium.

### CROSS REFERENCE TO RELATED APPLICATIONS

This application claims the benefit of priority pursuant to 35 U.S.C. §119(e)(1) from provisional patent application Serial No. 60/442,480 filed Jan. 27, 2003.

### FIELD OF THE INVENTION

This invention relates to methods and apparatus for electrically assisting the normal contraction of the heart.

### BACKGROUND

Congestive heart failure is characterized by the gradual inability of the heart to maintain optimal circulation to the body and its representative organs. Common symptoms of congestive heart failure include difficulty in breathing, chest discomfort, profound fatigue, and swelling of the ankles.

Congestive heart failure typically develops in an insidious pattern. In initiation of this maldevelopment, the heart cell, known as a cardiomyocyte, begins to lose its ability to contract and reset itself in a linear pattern. The subsequent inability of the cardiomyocyte to respond to physiologic demand sets in motion a deleterious cascade of compensatory physiologic phenomena.

Current clinical treatements include drug therapy, surgery, restraint devices, and electrical stimulation. Current drug therapy includes aspirin, beta blockers, dihydropyridine and mixed calcium channel blockers, thiazide and loop diuretics, digitalis, angiotensin converting enzyme inhibitors, nitrates, angiotensin two converting enzyme receptor blockers, human B-type natriuretic peptide infusion, aldosterone antagonists, and others. By its inherent nature, pharmacologic interventions will continue to advance safely but slow. Drug therapy has made little, if any, headway in the overall incidence and prevalence of congestive heart failure.

Surgical methods for addressing advanced failure related states and postinfarction phenomena include ventriculectomy procedures. These procedures involve surgical reduction of nonviable and/or marginally viable myocardium, rendering the heart physically and functionally smaller. The Batista operation specifically attempts immediate geometric remodeling of the failing myocardium with mixed results.

Another treatment modality for congestive heart failure involves the use of restraint devices. These devices restrain further mechanical degradation of the cardiomyocyte in established failure states but do not address autonomic insufficiency (electrical degradation).

Heart transplantation will likely remain superior to this approach but is limited by a continued disparity in donated hearts, insurance coverage, and rejection issues.

Left Ventricular Assist Devices are a highly promising emerging field which are very expensive and necessarily plagued with a multitude of moving parts. Lifelong permanent anticoaglation with warfarin will be necessary for many of these patients.

Electrical pacing of the heart has now reached a worldwide industry standard. Pacing began as an isolated single electrical stimulus designed to overdrive fatal bradycardia, a dangerously low heart rate. Understanding of the deterioration of the QRS interval in failure states has led to further innovation in addressing electromechanical failure. Current biventricular pacing modalities seek to correct this delay.

What is clearly needed, therefore, is improved methods and apparatus to more completely augment the cardiac functioning of patients suffering from congestive heart failure.

### SUMMARY

### BRIEF DESCRIPTION OF THE DRAWINGS

FIG. 1 is a frontal view of the pericardial augmentation apparatus encircling the left and right ventricles of the heart.

FIG. 2 is a frontal view of the pericardial augmentation apparatus encircling the left and right ventricles and the left and right atria of the heart.

FIG. 3 is a cross section of the pericardial augmentation apparatus.

### DETAILED DESCRIPTION

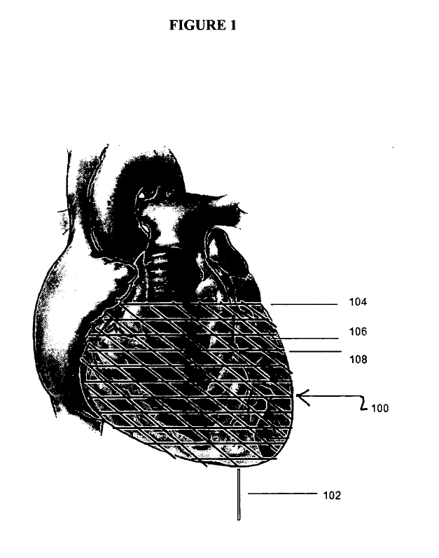

FIG. 1 shows the pericardial augmentation apparatus 100 which is made of a flexible mesh material that encircles the left and right ventricles of the heart. The pericardial augmentation apparatus 100 is connected at the apex (unnumbered) to a control cable 102 which is electrically connected to an implanted stimulator (not shown). The pericardial augmentation apparatus 100 is placed over the desired portion of the heart and is adjusted to closely fit the varying external contours of individually shaped hearts by tightening drawstrings 104 and pursestrings 106. Following shaping the pericardial augmentation apparatus 100 to the heart, micronibs 108 located at the conjunction of individual mesh strands (unnumbered) contact the exterior surface of the heart and help to hold the pericardial augmentation apparatus 100 in place.

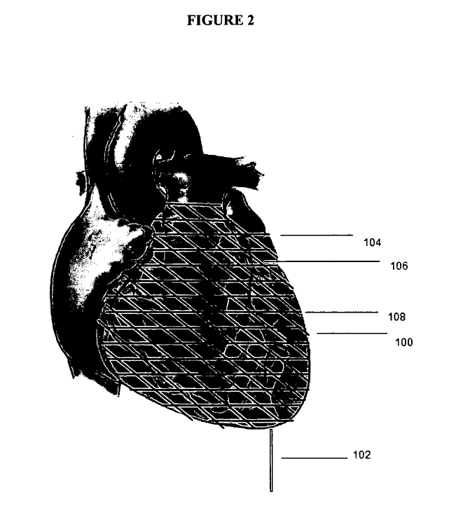

FIG. 2 shows that the pericardial augmentation apparatus 100 is capable of encircling the left and right ventricles as well as the left and right atria of the heart.

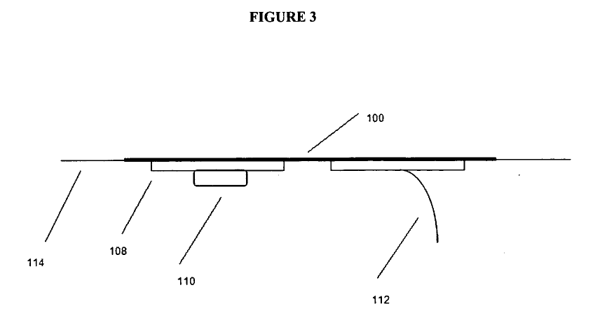

FIG. 3 shows that a plurality of micronibs 108 extend from the backside (unnumbered) of the pericardial augmentation apparatus 100 to contact the exterior surface of the heart. The micronibs may comprise various shapes, including but not limited to spherical endings 110 and/or pronged endings 112. A signal wire 114 is located within the individual strands of mesh material (unnumbered) comprising the pericardial augmentation apparatus 100.

The pericardial augmentation apparatus 100 is inserted into a small incision at the apex of the pericardial sac. The pericardial augmentation apparatus 100 is then advanced to the base of the heart and secured to the pericardial sac by sutures. A loose loop around the artial appendage and great vessels of the heart further secures the pericardial augmentation apparatus 100. The pericardial augmentation apparatus 100 engages the epicardium with barbed titanium micronibs 108 that also function to secure the pericardial augmentation apparatus 100. The micronibs 108 do not violate the epicardial vessels. All incisions are sealed for patency.

The control cable 102 connects the mesh (unnumbered) to an implanted stimulator (not shown) containing control microprocessors. A plurality of dedicated micronibs 108 is connected to the control cable 102 within the mesh (unnumbered) allows for efferent and afferent applications of electrical stimulus to the heart. The control device analyzes the electrical signals from the heart and provides the necessary electrical stimulation for each dedicated micronib 108. The electrical stimulation is therefore synchronized with the normal contraction of the heart, thereby matching the normal electrical pattern to assist the heart, rather than being a driving force.

What is claimed is:

1. An apparatus for targeting and coordinating a sequential propagation of synchronized current with autonomic propagation to maximize ejection fraction, comprising:
a. a mesh grid having crossing strands;
b. a plurality of micronibs attached to the mesh grid;
c. a control cable electrically connected to the micronibs; and
d. a control device electrically connected to the control cable to conduct synchronized electrical energy to separate portions of the heart via the micronibs to augment the normal contraction of the heart.

2. The apparatus of claim 1 further comprising drawstrings and pursestrings to adjust the size of the mesh grid to conform to the external contours of the heart.
